(19) United States
(12) Patent Application Publication
(10) Pub. No.: US 2014/0271903 A1
(43) Pub. Date: Sep. 18, 2014

(54) USE OF THERMO-SENSITIVE GEL FOR CONTROLLED DELIVERY OF ALK-5 INHIBITORS TO THE EYE AND RELATED METHODS

(71) Applicant: NORTHEAST OHIO MEDICAL UNIVERSITY, Rootstown, OH (US)

(72) Inventors: Vijaykumar Sutariya, Tampa, FL (US); Hiroshi Nakamura, Hudson, OH (US); Werner Geldenhuys, Kent, OH (US)

(21) Appl. No.: 13/803,585

(22) Filed: Mar. 14, 2013

(51) Int. Cl. A61K 47/34; A61K 9/51; A61K 31/4439

(52) U.S. Cl. CPC A61K 47/34; A61K 31/4439; A61K 9/5153
USPC 424/501; 514/772.1

(57) ABSTRACT

The present invention relates to a methods for extending the period of filtering bleb survival and/or providing for long term bleb survival following Glaucoma Filtration Surgery by delivering an ALK-5 inhibitor to a wound area (the surgical site) of a patient's eye. More particularly, the present invention relates to a method for the controlled delivery of an ALK-5 inhibitor to patient's eye using a thermo-sensitive polymer formulation, wherein the ALK-5 inhibitor is first contained in the polymer formulation at a temperature sufficient to maintain the formulation as a liquid and then applied to the eye wound opening, wherein the formulation turns to a gel. The use of the thermo-sensitive gel with ALK-5 inhibitor contained therein, provides for longer term bleb survival following Glaucoma Filtration Surgery (GFS) on a patient's eye. Thus, the present invention is particularly effective in inhibiting ocular fibrotic wound response following GFS.

### FIELD OF THE INVENTION

The present invention relates to a method for extending the period of filtering bleb survival and/or providing for long term bleb survival following Glaucoma Filtration Surgery by delivering an ALK-5 inhibitor to a wound area (the surgical site) of a patient's eye. More particularly, the present invention relates to a method for the controlled delivery of an ALK-5 inhibitor to patient's eye using a thermo-sensitive polymer formulation, wherein the ALK-5 inhibitor is first contained in the polymer formulation at a temperature sufficient to maintain the formulation as a liquid and then applied to the eye wound opening, wherein the formulation turns to a gel. The use of the thermo-sensitive gel with ALK-5 inhibitor contained therein, provides for longer term bleb survival following Glaucoma Filtration Surgery (GFS) on a patient's eye. Thus, the present invention is particularly effective in inhibiting ocular fibrotic wound response following GFS.

### BACKGROUND OF THE INVENTION

Glaucoma is a leading cause of blindness in the United States. It is estimated that, in 2010, there were about 60.5 million people with open angle glaucoma and angle closure glaucoma. In this type of glaucoma, blockage of the sponge-like trabecular meshwork (“TM”) of the eye slows drainage of the aqueous humor, increasing the intraocular pressure (“IOP”) and causing damage to the eye. Glaucoma Filtration Surgery (“GFS”), also referred to as a Trabeculectomy, is an often recommended treatment for these types of glaucoma.

In GFS, the surgeon attempts to bypass the eye's natural drainage system by creating a new drainage pathway through which the aqueous humor can leave the eye. Although there are many variations, the GFS surgery generally involves the following procedure. The eye is rotated downward to expose the conjunctiva and sclera above the cornea and a small incision is made in the conjunctiva, which is then carefully lifted and separated from the sclera. A half thickness flap of the sclera is then dissected up to the edge of the cornea. At the base of the flap, a small piece of tissue consisting of the sclera, TM and/or cornea is removed to create an opening into the anterior chamber of the eye through which fluid may leak, thus lowering the intraocular pressure. The flap of sclera is then sutured down and these sutures function by controlling the amount of fluid allowed to escape from the anterior chamber of the eye. The conjunctiva is then sutured back into place to create a waterproof seal. After the surgery, the fluid flowing out of the anterior chamber through the opening site will collect between the conjunctiva and the sclera to form a blister of fluid referred to as a filtering bleb from where it can be reabsorbed into the body.

Unfortunately, the body's ocular fibrotic wound response, more commonly known as post-operative scarring, is a major cause of impaired vision and blindness, especially as a consequence of GFS. For example, in recent rabbit studies, it has been found that, following GFS, aggressive scarring produces early bleb failure, typically within 4 to 14 days after GFS. Such scarring can cause the outflow from the anterior chamber to again become blocked and the surgery to fail.

To prevent such scarring, an anti-scarring treatment is usually required. Such treatments include attempting to prevent bleb failure using antimetabolites. Generally, antimetabolites, such as 5-fluorouracil (5-FU) or mitomycin-C (MMC), are commonly used to reduce post-operative scarring at the site of GFS. While recent rabbit studies have shown these antimetabolites, particularly MMC, to be effective in preventing such post-operative scarring by extending bleb survival for around 30 days, however, these agents are cytotoxic and are believed to cause widespread cell death. Hence, these agents have also been associated with severe and potentially blinding complications.

More recently, ALK-5 inhibitors have been described in several publications to be effective suppressors of downstream proteins of transforming growth factor (TGF-β). In previous experiments, it was found that at least one ALK-5 inhibitor, namely a hydrochloride hydrate, available from Sigma Aldrich, Saint Louis, Mo., under the tradename SB-505124, induced a marked reduction in downstream proteins of transforming growth factor (TGF-β), connective tissue growth factor (CTGF) and a-smooth muscle actin (a-SMA), in cultured rabbit subconjunctival fibroblasts.

In addition, in an in vivo rabbit GFS model, it was shown that SB-505124 extended the period of filtering bleb survival. Toxicity of SB-505124 in GFS was shown to be substantially lower than that of MMC. These results indicate that SB-505124 is able to suppress subconjunctival scarring via blocking of TGF-β activity without much tissue damage and with vastly reduced toxicity compared to MMC. On the other hand, while the bleb survival was improved by SB-505124 the survival period was substantially shorter compared to MMC.

Thus, the need exists for a method of extending the period of long term bleb survival without the cytotoxic side effects found in antimetabolites, like MMC. Further, given the potential effectiveness of SB-505124 and other ALK-5 inhibitors in extending the period of filtering bleb survival, the need exists for a method of maintaining an appropriate or adequate concentration of SB-505124 in filtering bleb, thereby extending the period of bleb survival. Still further, a need exists for inhibiting ocular fibrotic wound response following GFS by controlling the delivery of SB-505124.

Extended release drug delivery systems that utilize biodegradable polymers are well known in the art. Attempts to develop sustained-release formulations have included the use of a variety of biodegradable and non-biodegradable polymer (e.g. poly(lactide-co-glycolide)) microparticles containing the active ingredient (see e.g., Wise. et al., Contraception, 8:227-234 (1973); and Hutchinson et al., Biochem. Soc. Trans., 13:520-523 (1985)), and a variety of techniques are known by which active agents, e.g. proteins, can be incorporated into polymeric microspheres (see e.g., U.S. Pat. No. 4,675,189 and references cited therein).

Utilization of the inherent biodegradability of these materials to control the release of an active agent and provide a more consistent sustained level of medication provides improvements in the sustained release of active agents. Unfortunately, some of the sustained release devices utilizing microparticles still suffer from such things as: active agent aggregation formation; high initial bursts of active agent with minimal release thereafter; and incomplete release of active agent.

Other drug-loaded polymeric devices have also been investigated for long term, therapeutic treatment of various diseases, again with much attention being directed to polymers derived from alpha hydroxycarboxylic acids, especially lactic acid in both its racemic and optically active form, and glycolic acid, and copolymers thereof. These polymers are commercially available and have been utilized in FDA-approved systems, e.g., the Lupron Depot™, which consists of injectable microcapsules which release leuprolide acetate for about 30 days for the treatment of prostate cancer.

Various problems identified with the use of such polymers include: inability of certain macromolecules to diffuse out through the matrix; deterioration and decomposition of the drug (e.g., denaturation caused by the use of organic solvents); irritation to the organism (e.g. side effects due to use of organic solvents); low biodegradability (such as that which occurs with polycondensation of a polymer with a multifunctional alcohol or multifunctional carboxylic acid, i.e., ointments); and slow rates of degradation.

The use of polymers which exhibit reverse thermal gelation have also been reported. For example, Okada et al., Japanese Patent Application 2-78629 (1990) describe biodegradable block copolymers synthesized by transesterification of poly(lactic acid) (PLA) or poly(lactic acid)/glycolic acid (PLGA) and poly(ethylene glycol) (PEG). PEGs with molecular weights ranging from 200 to 2000, and PLA/GA with molecular weights ranging from 400 to 5000 were utilized. The resultant product was miscible with water and formed a gel. Biodegradable gels can be carriers for biologically active materials such as hormones, enzymes, antibiotics, antineoplastic agents, and cell suspensions.

Attempts have been made to provide systems for parenteral delivery of a drug comprising an injectable biodegradable block copolymeric drug delivery liquid having reverse thermal gelation properties. Specifically, these thermosensitive gels exist as a mobile viscous liquid at low temperatures, but form a rigid semisolid gel at higher temperatures. Thus, it is possible to use these polymers to design a formulation which is liquid at room temperature or at lower temperatures, but gels once injected, thus producing a depot of drug at the injection site. The systems described by Cha et al. utilize a hydrophobic A polymer block comprising a member selected from the group consisting of poly(alpha-hydroxy acids) and poly(ethylene carbonates) and a hydrophilic B polymer block comprising a PEG.

Use of extended release drug delivery systems using reverse thermo-sensitive triblock copolymers with a PLGA-PEG-PLGA structure have been studied as a delivery vehicle for proteins and peptides. Reverse thermo-sensitive triblock copolymers with a PLGA-PEG-PLGA structure have also been studied as a vehicle for drug loaded PLGA microspheres and administered intravitreally for the treatment of cytomegalovirus retinitis. The PLGA-PEG-PLGA gels has been successful to control the release up to 30 days for many drugs including gancylcovir, paclitaxel and insulin.

To date however, reverse thermo-sensitive triblock copolymers of PLGA-PEG-PLGA have not been used as an extended release delivery system for an ALK-5 inhibitor for the purpose of extending the period of filtering bleb survival and/or providing for long term bleb survival following Glaucoma Filtration Surgery.

### SUMMARY OF THE INVENTION

The present invention involves methods for extending the period of filtering bleb survival and/or providing for long term bleb survival following GFS on the eye of a patient by (i) preparing a formulation containing a thermo-sensitive polymer and an ALK-5 inhibitor; (ii) opening the eye of the patient who has had Glaucoma Filtration Surgery; and (iii) applying the formulation into the surgical site of Glaucoma Filtration Surgery, whereby the formulation is turned into a gel and controllably releases a concentration of the ALK-5 inhibitor sufficient to extend the period of filtering bleb survival following Glaucoma Filtration Surgery to more than 14 days.

Another embodiment of the present invention is directed to a method for providing for long term bleb survival following Glaucoma Filtration Surgery on the eye of a patient comprising: (i) preparing a liquid formulation containing a thermo-sensitive polymer and an ALK-5 inhibitor; (ii) opening the eye of the patient that has had Glaucoma Filtration Surgery; and (iii) applying the formulation into the wound area of the eye whereby the liquid formulation is turned into a gel and controllably releases a concentration of the ALK-5 inhibitor sufficient to provide for long term bleb survival following Glaucoma Filtration Surgery.

Another embodiment of the present invention is the method of paragraph [0017] or [0018] wherein the ALK-5 inhibitor is entrapped within the thermo-sensitive polymer.

Still another embodiment of the present invention is the method of any of paragraphs [0017] through [0019] wherein the liquid formulation is injectable and the formulation is injected into or onto the wound area of the eye.

Another embodiment of the present invention is the method of any of paragraphs [0017] through [0020] wherein the thermo-sensitive polymer is a poly(lactide-co-glycolide)-polyethylene glycol-poly(lactide-co-glycolide) block copolymer.

Another embodiment of the present invention is the method of any of paragraphs [0017] through [0021] wherein the formulation containing the poly(lactide-co-glycolide)-polyethylene glycol-poly(lactide-co-glycolide) block copolymer is in a liquid state below about 10° C.

Another embodiment of the present invention is the method of any of paragraphs [0017] through [0022] wherein the formulation containing the poly(lactide-co-glycolide)-polyethylene glycol-poly(lactide-co-glycolide) block copolymer is in a liquid state below about 4° C.

Another embodiment of the present invention is the method of any of paragraphs [0017] through [0023] wherein the formulation containing the poly(lactide-co-glycolide)-polyethylene glycol-poly(lactide-co-glycolide) block copolymer is in a gel state at the body temperature of the patient.

Another embodiment of the present invention is the method of any of paragraphs [0017] through [0024] wherein the ALK-5 inhibitor is a hydrochloride hydrate.

Another embodiment of the present invention is the method of any of paragraphs [0017] through [0025] wherein the ALK-5 inhibitor is contained within poly(lactide-co-glycolide) nanoparticles.

### BRIEF DESCRIPTION OF THE DRAWINGS

For a more complete understanding of the features and advantages of the present invention, reference is now made to the detailed description of the invention along with the accompanying figures in which:

FIG. 1 is a graph showing the sol-gel transition and gelation temperature ranges for PLGA-PEG-PLGA block co-polymers at different polymer concentrations.

FIG. 2 is a printout of a chromatogram taken of SB-505124 (5 μg/ml).

FIG. 3 is a graph showing the results of a control study measuring the IOP in the right eye of a rabbit treated with 30% weight to volume of a PLGA-PEG-PLGA polymer solution containing no drug over time.

FIG. 4 is a graph showing the results of a study measuring the IOP in the right eye of a rabbit treated with 30% weight to volume of a PLGA-PEG-PLGA polymer solution containing 10 mg of SB505124 over time. The left eye is used as the control.

FIG. 5 is a graph showing the results of a study measuring the IOP in the right eye of a rabbit treated with 30% weight to volume of a PLGA-PEG-PLGA polymer gel solution containing 10 mg of SB505124 over time. The left eye is used as a control.

FIG. 6 is a graph showing the results of a study measuring the average IOP in the right eye of a rabbit treated with 25% weight to volume of a PLGA-PEG-PLGA polymer solution containing 10 mg of SB505124 nanoparticles over time. The left eye is used as a control.

FIG. 7 is a graph showing the results of an in vitro drug release study for 25% weight to volume of a PLGA-PEG-PLGA polymer solution containing 10 mg SB-505124 at 37° C.

### DETAILED DESCRIPTION OF ILLUSTRATIVE EMBODIMENTS

The present invention involves a method for extending the period of filtering bleb survival and/or providing for long term bleb survival following GFS on the eye of a patient by application to the wound area of the eye of a formulation containing a thermo-sensitive polymer and an ALK-5 inhibitor. The formulation is a liquid when it is applied to the eye but turns into a gel when it reaches the body temperature of the patient. The polymer formulation controllably releases a concentration of the ALK-5 inhibitor sufficient to extend the period of filtering bleb survival following Glaucoma Filtration Surgery for more than 14 days.

The method of the present invention utilizes formulations that contain reverse thermo-sensitive polymers (“thermo-sensitive polymers”). Solutions containing these polymers are known to those of skill in the art and have the property of changing from a liquid state to a gel state within a specific temperature range. As used herein, the term “gel” refers to the semi-solid phase that spontaneously occurs as the temperature of the thermo-sensitive polymer solution is raised to or above the gelation temperature of the polymer. The terms “gelation temperature” and “reverse thermal gelation temperature” are used interchangeably herein and refer to the temperature at which the polymer solution undergoes reverse thermal gelation, i.e. the temperature below which the polymer solution is a liquid and above which the polymer solution undergoes phase transition to increase in viscosity or to form a semi-solid gel.

The thermo-sensitive polymers used in the method of the present invention are preferably biodegradable. As used herein, “biodegradable” means that the block copolymer can chemically break down or degrade within the body into components that are well tolerated by the body of the patient. The rate of degradation can be the same or different from the rate of drug release.

The thermo-sensitive polymer may be a PLGA-PEG-PLGA triblock copolymer and may be synthesized by ring opening co-polymerization as described by Ghahremankhani et al., (Ghahremankhani A A, Dorkoosh F, Dinarvand R, PLGA-PEG-PLGA tri-block copolymers as an in-situ gel forming system for calcitonin delivery, Polymer Bulletin, 2007, 59, 637-646) the disclosure of which is incorporated herein by reference, and as set forth in Example 1 below. Suitable PLGA-PEG-PLGA triblock copolymers are also commercially available from PolySciTech (a Division of Akina, Inc.) of West Lafayette, Ind.

Thermo-sensitive PLGA-PEG-PLGA triblock copolymers are well described in the art. These polymers may have molecular weights from about 2000 Da to about 40,000 Da, but may have any molecular weight provided that it has the required reverse thermo-sensitive properties. In some embodiments, the PLGA-PEG-PLGA triblock polymers may have a molecular weight of about 30,000 Da. In some other embodiments, the PLGA-PEG-PLGA triblock polymers may have a molecular weight of about 4000 Da. In some other embodiments, the PLGA-PEG-PLGA triblock copolymer may have a molecular weight distribution of 1500:1000:1500 but as one of ordinary skill in the art should appreciate, other molecular weight distributions may be possible and are within the scope of the present invention. Nor is the ratio of lactic acid to glycolic acid in the PLGA segments of the polymer critical provided that the polymer has the required reverse thermo-sensitive properties. In some embodiments, the PLGA segment of the copolymer has a lactic acid to glycolic acid ration of about 1:1 and in some other embodiments, the PLGA segment of the copolymer has a lactic acid to glycolic acid ration of about 3:1.

For ease of administration, the formulation containing the thermo-sensitive polymer should, as set forth above, be in a liquid state prior to being administered to the patient. By the time it has reached the body temperature of the patent, the formulation should have transformed into its gel state thereby entrapping the ALK-5 inhibitor within the polymer gel, and facilitating the controlled, i.e. extended, release of the ALK-5 inhibitor to the eye as the polymer breaks down over time. In some embodiment the thermo-sensitive polymer is in a liquid state at or below about 4° C. In some other embodiments, the thermo-sensitive polymer is in a liquid state at or below about 10° C. It is contemplated, therefore, that the gelation temperature of the polymer formulation should fall be between the temperature of the polymer formulation at the time of administration and the body temperature of the patient, and is preferably close to, but less than, the body temperature of the patent. In some embodiments, the gelation temperature of the polymer is from about 32° C. to about 42° C. See FIG. 1.

In some embodiments, the formulation containing the thermo-sensitive polymer and ALK-5 inhibitor may be prepared by dissolving the polymer in water or a buffer at or about 4° C. at a concentration of from about 15% w/v to about 40% w/v of polymer. It has been found that 25-30% w/v mixtures give a good reverse thermo-sensitive polymer solution. In some embodiments, a 25% w/v polymer solution was used. In some other embodiments, a 30% w/v polymer solution was used It has been found that adding the polymer to the water or buffer (rather than adding the water of buffer to the polymer) prevents or at least limits clumping of the polymer. To arrive at the formulation used with the method of the present invention, the polymer solution is then mixed with a therapeutically significant amount of an ALK-5 inhibitor to produce the formulation of the present invention.

As set forth above, ALK-5 inhibitors have been shown to reduce ocular scarring and extend significantly the period of filtering bleb survival following GFS and to induce a marked reduction in downstream proteins of transforming growth factor (TGF-β), including connective tissue growth factor (CTGF) and a-smooth muscle actin (a-SMA), in cultured rabbit subconjunctival fibroblasts. ALK-5 inhibitors are also sometimes described as a TGF-13 receptor 1.

One suitable ALK-5 inhibitor is a hydrochloride hydrate, available from Sigma Aldrich (St. Louis, Mo.), under the tradename SB-505124. It has the empirical formula C₂₀H₂₁N₃O₂.xHCl.yH2O and the following structure:

[chemical structure]

A representative chromatogram of SB-505124 (5 μg/ml) is shown as FIG. 2.

Further, the ALK-5 inhibitor, SB-505124, has been found to be less cytotoxic and causing less cell death than antimetabolites, such as 5-fluorouracil (5-FU) or mitomycin-C (MMC). See e.g. Sapitro, et al. “Suppression of transforming growth factor-β effects in rabbit subconjunctival fibroblasts by activin receptor-like kinase 5 inhibitor” Molecular Vision 2010; 16:1880-1892, the disclosure of which is incorporated herein by reference. Further, it should be appreciated that the results of the in vivo release tests shown in FIGS. 3-6 and discussed in detail below, were reached without any significant sign of inflammation or toxicity reactions at the sites of gel administration.

Dosage amounts of the ALK-5 inhibitor SB-505124 ranging from 5 to 20 mg have been found to be effective for the prevention of scarring in the in vivo rabbit GFS models. In one embodiment of the invention the therapeutically significant amount of SB-505124 was 10 mg. In some embodiments, the concentration of SB-505124 is from about 1 mg to about 5 mg of SB-505124 in 0.2 mL of the final formulation to be applied into the eye.

As used herein, the terms “polymer formulation,” “gel formulation,” “drug delivery liquid” or “drug delivery liquid having reverse thermal gelation properties” shall mean a formulation that contains a thermo-sensitive polymer having reverse thermal gelation properties and a drug (the drug per se can either be dissolved or colloidal) suitable for administration to a warm-blooded animal and which forms a gelled drug depot when the temperature is raised to or above the gelation temperature of the thermo-sensitive block copolymer.

In some embodiments, the ALK-5 inhibitor may be dissolved and/or suspended directly into the polymer solution, as set forth above. In other embodiments, however, the ALK-5 inhibitor is entrapped within nanoparticles of PLGA prepared by the solvent evaporation method and then suspended in the polymer. Nanoparticles have the advantage of adding several beneficial features to the delivery system, in part by slowing down the possible breakdown of the ALK-5 inhibitor by, for instance, hydrolysis. The inclusion of nanoparticles into the gel may increase the therapeutic window of the drug delivery system.

Detailed techniques for making nanoparticles using the solvent evaporation method are well known in the art. Generally, the nanoparticles may be made by dissolving the ALK-5 inhibitor into an organic solvent such as dimethylsulfoxide, ethylacetate, dichloromethane, acetonitrile, chloroform, diethylether, or acetone. This drug solution is then dripped into a stirring buffer solution such as phosphate buffer saline (pH range of 7.2 to 8), or 0.9% NaCl (saline), or Tris-buffered saline (pH range 7.2 to 8). The solution is allowed to stir for several hours until the organic solvent has evaporated. The nanoparticles can then be concentrated using centrifugation methods or cleaned using Sepharose G-25 or G-50 column chromatography. The size of the nanoparticles formed using this methods may be determined using dynamic light scattering and ordinarily ranges from about 50 nm to about 500 nm with an average size of about 150 nm in diameter, but these sizes are in no way limiting.

As set forth above, the present invention includes methods of for extending the period of filtering bleb survival and/or providing for long term bleb survival following GFS on the eye of a patient, which may include some or all of the following steps. First, a drug delivery formulation containing a thermo-sensitive polymer having a gelation temperature close to but less than the body temperature of the patient and a therapeutic amount of an ALK-5 inhibitor such as of SB-505124 is prepared as set forth herein. The formulation is kept at a temperature below the gelation temperature to ensure that the formulation remains in a liquid state until use.

Second, an eye of a patient having recently undergone GFS is held open in such a way as to expose the wound area of the patient's eye and the formulation is administered to the wound area of the patient's eye at a temperature below the gelation temperature for the polymer. In some embodiments, the liquid polymer formulation is injected into the wound area of the eye. However, even in its liquid state, the polymer formulation may be relatively viscous and, in some embodiments, the liquid polymer formulation may be applied into the wound area of the eye using a spatula or similar applicator. As the polymer formulation reaches the body temperature of the patient, the thermo-sensitive polymer in the solution reaches its gelation temperature and becomes a gel, encapsulating the ALK-5 inhibitor and serving as a depot for its extended release into the eye as the polymer degrades over time. It has been found that the extended release of the ALK-5 inhibitor from the polymer gel extends the period of filtering bleb survival and/or provides for long term bleb survival following GFS.

In in vivo rabbit glaucoma filtration studies, for example, the formulation showed suppression of scarring and bleb survival for prolonged period of time (over 20 days). (See Example 8) The bleb survival was around 1 week for control while it was over 20 days for 30% w/v thermo reversible gel of SB505124. (See FIGS. 3-6) Moreover, as shown in FIGS. 4-6, the inter ocular pressure (“IOP”) of the surgically operated eye treated with polymer formulation containing the ALK-5 inhibitor was consistently lower than the control eye further indicating improved filtering bleb survival.

In light of the foregoing, it should be appreciated that the present invention significantly advances the art by providing an improved method for extending the period of filtering bleb survival and/or providing for long term bleb survival following Glaucoma Filtration Surgery on the eye. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

### EXAMPLES

The following examples are offered to more fully illustrate the invention, but are not to be construed as limiting the scope thereof. Further, while some of examples may include conclusions about the way the invention may function, the inventor do not intend to be bound by those conclusions, but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Example 1

### Synthesis of the PLGA-PEG-PLGA Copolymer

PLGA-PEG-PLGA copolymer was synthesized through a ring opening copolymerization as described by Ghahremankhani et al., 2007 (Ghahremankhani AA, Dorkoosh F, Dinarvand R, Polymer Bulletin, 2007, 59, 637-646), the disclosure of which is hereby encorporated by reference in its entirety. PEG 1500 was loaded to a stainless steel reactor and heated for 2 hours at 150° C. at 5 mmHg vacuum. D,L-lactide (19.9 g) and glycolide (5.7 g) was melted at the same temperature for 30 min and stirred. Stannous 2-ethylhezanoate (0.04 g) was added as catalyst and heating was continued at 160° C. for 6 h under 5 mmHg vacuum. After the completion of reaction, the copolymer was dissolved in cold water (4° C.) and was heated to 80° C. to precipitate to remove impurities and unreacted material for purification. The resultant triblock polymer was of the polymer composition PLGA-PEG-PLGA. The approximate ratio of PLGA to PEG was 50:25 in the polymer.

### Example 2

### Preparation of Formulation Containing PLGA-PEG-PLGA COPOLYMER 25% w/v And an ALK-5 Inhibitor

A formulation containing 25% w/v PLGA-PEG-PLGA co-polymer and 10 mg of the ALK-5 inhibitor SB-505124 was prepared by first dissolving 200 mg of the PLGA-PEG-PLGA copolymer of Example 1 in 1 ml of water at 4° C. and vortex mixing it for 10 minutes until the polymer is fully dissolved. Finally, 10 mg of SB-505124 powder was slowly added to the cold (4° C.) polymer solution and mixed. The dispersion was kept in the refrigerator (6-12 hours) until a suspension/solution was formed.

### Example 3

### Preparation of Formulation Containing PLGA-PEG-PLGA Copolymer 25% w/v and Nanoparticles Containing an ALK-5 Inhibitor

A formulation containing 25% w/v PLGA-PEG-PLGA co-polymer and 10 mg of the ALK-5 inhibitor SB-505124 was prepared by first dissolving 200 mg of the PLGA-PEG-PLGA copolymer of Example 1 in 1 ml of water at 4° C. and stirring with magnetic stir bar. PLGA nanoparticles of an approximate MW of 8000 and containing the ALK-5 inhibitor SB-505124 were prepared from a 50:50 mixture of D,L-lactide and glycolide using the solvent evaporation method. The polymer and 10 mg of SB-505124 were dissolved in 3 ml acetone and the solution was added dropwise into a stirring solution of a phosphate buffer saline The solution was then stirred at 300 rpm for at least 2 hours. The nanoparticles were centrifuged for 30 minutes at 20,000×g, at 4° C. The liquid suspension containing the nanoparticles was added to a stirring 25% w/v polymer solution and suspended therein.

### Example 4

### Preparation of Formulation Containing PLGA-PEG-PLGA Copolymer 30% w/v and the ALK-5 Inhibitor

A formulation containing 30% w/v PLGA-PEG-PLGA co-polymer and 10 mg of the ALK-5 inhibitor SB-505124 was prepared by first dissolving 300 mg of the PLGA-PEG-PLGA copolymer of Example 1 in 1 ml of water at 4° C. and vortex mixing it for 10 minutes until the polymer is fully dissolved. Finally, 10 mg of SB-505124 powder was slowly added to the cold (4° C.) polymer solution and mixed. The dispersion was kept in the refrigerator (6-12 hours) until a suspension/solution was formed.

### Example 5

### Measurement of Gelation Temperature

The solution-gel transition and gelation temperature were studied by visual observation method. Various gel formulations were prepared using different concentration of polymer in water. For example, 10% w/v, 15% w/v, 20% w/v, 25% w/v, 30% w/v and 35% w/v. the sol-gel transition was visually determined by heating the solution from 4° C. to 70° C. The results of the study are reported on FIG. 1. The 25% w/v PLGA-PEG-PLGA formulation showed complete gel formation around 37° C. with stable viscosity.

### Example 6

### In Vitro Drug Release Study of the Gel

In vitro drug release was studied using a dialysis cassette method. 0.5 mL of the 25% (w/v) PLGA-PEG-PLGA polymer formulation containing 10 mg SB-505124 was injected into a dialysis cassette with a 10,000 MW cut-off at 4 degrees Celsius. The cassette is then placed into a 200 mL beaker containing 60 mL phosphate buffered saline pH 7.4 and stirred with a magnetic stir bar. The drug was released into 60 mL of water at 37 C, stirring at 30 rpms (n=3). 1 ml buffer samples were taken from the beaker at different time intervals and 1 mL of buffer was replaced with fresh buffer solution. The HPLC method was used for analysis of the samples which was consisted of C18 column of 150×4.6 mm and an inner diameter of 5 microns. The mobile phase consisted of a mixture of 0.1% v/v trifluoroacetic acid in water and 0.1% v/v trifluoroacetic acid in acetonitrile, using a mix ratio of 40/60 at a 1 ml/min flow rate. The samples were measured using HPLC with UV detection at 315 nm. The results of the in vitro study are shown as FIG. 7.

### Example 7

### Development of HPLC Method SB-505124

The HPLC method used a C18 column of 150×4.6 mm and an inner diameter of 5 microns. The mobile phase consisted of a mixture of 0.1% v/v trifluoroacetic acid in water and 0.1% v/v trifluoroacetic acid in acetonitrile, using a mix ratio of 40/60 at a 1 ml/min flow rate. The samples were measured using HPLC with UV detection at 315 nm. A representative chromatogram of SB-505124 (5 μg/ml) is shown at FIG. 2.

### Example 8

### BLEB Longevity Assay for 30% w/v PLGA-PEG-PLGA Gel Containing 10 MG SB-505124

Glaucoma filtration surgery (GFS) was performed on one eye of 4 rabbits. The 30% w/v PLGA-PEG-PLGA polymer formulation of Example 4 containing 10 mg of SB-505124 was applied to the wound area of rabbit numbers 1 and 2 and a formulation prepared in the manner of Example 4 but without the SB-505124, was applied to the wound area of rabbit numbers. 3 and 4. Because of the high viscosity of the liquid polymer formulation, it was applied to the wound area of the rabbits' eyes using a spatula. However, it is believed that the liquid polymer formulation could also have been injected into the wound area of the eyes using a properly sized syringe.

The intraocular pressures (IOPs) of the eyes was measured on a daily basis and the condition of the rabbit blebs was observed weekly. FIG. 3 is a graph showing the intraocular pressures (IOPs) of the right and left eyes in the control rabbits. GFS using the polymer formulation without SB-505124 was performed on the right eyes of these rabbits and the left eye is untreated control. As can be seen in FIG. 3, the IOP in the right eye of one of these control rabbits was much lower than that of the left eye for the couple of days after surgery, but became similar from day 5 and all blebs in the control eyes had failed within a week after the surgery.

FIGS. 4 and 5 are graphs showing the intraocular IOPs of the right and left eyes in rabbit numbers 1 and 2, respectively, which received the 30% w/v PLGA-PEG-PLGA formulation containing 10 mg SB-505124. As can be seen in FIGS. 4 and 5, the blebs in the eyes treated with the 30% w/v PLGA-PEG-PLGA formulation containing 10 mg SB-505124 survived until day 16 and 20 ((log rank=11.801, p<0.01, Kaplar Mier Analysis), indicating that formulation released the drug slowly and prevented the scarring in the eye up to day 20.

### Example 9

### BLEB Longevity 25% w/v PLGA-PEG-PLGA Gel Containing 10 MG SB505124 Nanoparticles (N=2)

Glaucoma filtration surgery (GFS) was performed on the right eye of a rabbit. The 25% w/v PLGA-PEG-PLGA formulation containing 10 mg of SB-505124 in nanoparticles of Example 3 was applied to the wound area of the right eye of the rabbit following GFS. Again, because of the high viscosity of the liquid polymer formulation, it was applied to the wound area of the rabbits' eyes using a spatula. However, it is believed that the liquid polymer formulation could also have been injected into the wound area of the eyes using a properly sized syringe. The intraocular pressures (IOPs) of the eyes were measured on a daily basis and the condition of the rabbit blebs was observed weekly. FIG. 6 is a graph showing the intraocular pressures (IOPs) of the right and left eyes of the rabbit after GFS.

What is claimed is:

1. Method for extending the period of filtering bleb survival following Glaucoma Filtration Surgery on the eye of a patient comprising:
(i) preparing a formulation containing a thermo-sensitive polymer and an ALK-5 inhibitor; and
(ii) opening the eye of the patient who has had Glaucoma Filtration Surgery and applying the formulation into the wound area of the eye, whereby the formulation is turnes into a gel and controllably releases a concentration of the ALK-5 inhibitor sufficient to extend the period of filtering bleb survival following Glaucoma Filtration Surgery.

2. The method of claim 1 wherein the ALK-5 inhibitor is entrapped within the thermo-sensitive polymer.

3. The method of claim 1 wherein the thermo-sensitive polymer is a poly(lactide-co-glycolide)-polyethylene glycol-poly(lactide-co-glycolide) block copolymer.

4. The method of claim 3 wherein the formulation containing the poly(lactide-co-glycolide)-polyethylene glycol-poly(lactide-co-glycolide) block copolymer is in a liquid state below about 10° C.

5. The method of claim 3 wherein the formulation containing the poly(lactide-co-glycolide)-polyethylene glycol-poly(lactide-co-glycolide) block copolymer is in a liquid state below about 4° C.

6. The method of claim 3 wherein the formulation containing the poly(lactide-co-glycolide)-polyethylene glycol-poly(lactide-co-glycolide) block copolymer is in a gel state at the body temperature of the patient.

7. The method of claim 1 wherein the ALK-5 inhibitor is a hydrochloride hydrate.

8. The method of claim 1 wherein the ALK-5 inhibitor is contained within poly(lactide-co-glycolide) nanoparticles.

9. The method of claim 1 wherein the formulation is injected into the wound area of the eye.

10. Method for providing for long term bleb survival following Glaucoma Filtration Surgery on the eye of a patient comprising:
(i) preparing a formulation containing a thermo-sensitive polymer and an ALK-5 inhibitor; and
(ii) opening the eye of the patient that has had Glaucoma Filtration Surgery and applying the formulation to the wound area of the eye whereby the formulation is turned into a gel that controllably releases a concentration of the ALK-5 inhibitor sufficient to provide for long term bleb survival following Glaucoma Filtration Surgery.

11. The method of claim 10 wherein the ALK-5 inhibitor is entrapped within the thermo-sensitive polymer

12. The method of claim 10 wherein the thermo-sensitive polymer is a poly(lactide-co-glycolide)-polyethylene glycol-poly(lactide-co-glycolide) block copolymer.

13. The method of claim 12 wherein the formulation containing the poly(lactide-co-glycolide)-polyethylene glycol-poly(lactide-co-glycolide) block copolymer is in a liquid state below about 10° C.

14. The method of claim 12 wherein the formulation containing the poly(lactide-co-glycolide)-polyethylene glycol-poly(lactide-co-glycolide) block copolymer is in a liquid state below about 4° C.

15. The method of claim 12 wherein the formulation containing the poly(lactide-co-glycolide)-polyethylene glycol-poly(lactide-co-glycolide) block copolymer is in a gel state at the body temperature of the patient.

16. The method of claim 10 wherein the ALK-5 inhibitor is a hydrochloride hydrate.

17. The method of claim 10 wherein the ALK-5 inhibitor is contained within poly(lactide-co-glycolide) nanoparticles.

18. The method of claim 10 wherein the formulation is injected into the wound area of the eye.
